(19) United States
(12) Patent Application Publication
(10) Pub. No.: US 2021/0267908 A1
(43) Pub. Date: Sep. 2, 2021

(54) PHARMACEUTICAL COMPOSITIONS OF RIVAROXABAN

(71) Applicant: MANKIND PHARMA LTD., New Delhi (IN)

(72) Inventors: Rajnish Kumar SINGH, Manesar (IN); Tarun GARG, Manesar (IN); Muthulingam CHITHAMBARAM, Manesar (IN); Anil KUMAR, Manesar (IN)

(21) Appl. No.: 17/188,401

(22) Filed: Mar. 1, 2021

(30) Foreign Application Priority Data
Mar. 2, 2020 (IN) 202011008807

(51) Int. Cl. A61K 9/51; A61K 31/5377; A61J 3/07

(52) U.S. Cl. CPC A61K 9/51; A61J 3/07; A61K 9/5192; A61K 31/5377

(57) ABSTRACT

There is provided a dosages form comprising rivaroxaban and one or more pharmaceutically acceptable excipients. The present invention also provides a stable capsule dosage form comprising rivaroxaban and one or more pharmaceutically acceptable excipients. The invention also relates to process of preparation of such compositions.

### FIELD OF INVENTION

The present invention provides a stable capsule composition comprising rivaroxaban and one or more pharmaceutically acceptable excipients. The invention also relates to process of preparation of such compositions and their use to treat thromboembolic disorders.

### BACKGROUND OF INVENTION

Rivaroxaban, a factor Xa (FXa) inhibitor, is the active ingredient in XARELTO™ tablets with the chemical name 5-Chloro-N-({(5S)-2-oxo-3-[4-(3-oxo-4-morpholinyl)phenyl]-1,3-oxazolidin-5-yl}methyl)-2-thiophenecarboxamide. The molecular formula of rivaroxaban is C₁₉H₁₈ClN₃O₅S and the molecular weight is 435.89. The structural formula is:

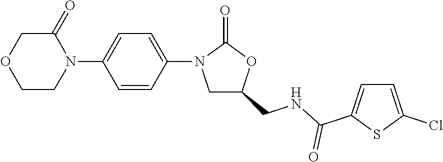 [SMILES: O=C(NC[C@H]1CN(c2ccc(N3CCOCC3=O)cc2)C(=O)O1)c1ccc(Cl)s1]

Rivaroxaban is marketed under the trade name XARELTO™ in United States as 2.5 MG, 5 MG, 10 MG and 20 MG tablets for the treatment to reduce the risk of stroke and systemic embolism in patients with non-valvular atrial fibrillation, for the treatment of deep vein thrombosis (DVT), for the treatment of pulmonary embolism (PE), for the reduction in the risk of recurrence of DVT and/or PE in patients at continued risk for recurrent DVT and/or PE after completion of initial treatment lasting at least 6 months, for the prophylaxis of DVT, which may lead to PE in patients undergoing knee or hip replacement surgery, for the prophylaxis of venous thromboembolism (VTE) in acutely ill medical patients at risk for thromboembolic complications not at high risk of bleeding, in combination with aspirin, to reduce the risk of major cardiovascular events (cardiovascular (CV) death, myocardial infarction (MI) and stroke) in patients with chronic coronary artery disease (CAD) or peripheral artery disease (PAD).

Rivaroxaban is a pure (S)-enantiomer. It is an odorless, non-hygroscopic, white to yellowish powder. Rivaroxaban is only slightly soluble in organic solvents (e.g. acetone, polyethylene glycol 400) and is practically insoluble in water and aqueous media with pH 1-9. An amount of approximately 5-7 mg/L rivaroxaban is pH-independently soluble in aqueous media at 25° C. The partition coefficient in octanol/water (log Po/w) is 1.5. It belongs to BCS class II. Its low aqueous solubility is overcome by reducing the particle size with micronization.

There are several patent literatures known in the art which disclose the pharmaceutical compositions of rivaroxaban with one or more pharmaceutically acceptable excipients.

Rivaroxaban as a product and its use for treating thromboembolic disorders is disclosed in U.S. Pat. No. 7,157,456 B2; U.S. Pat. No. 7,585,860 B2 and U.S. Pat. No. 7,592,339 B2 assigned to Bayer HealthCare.

U.S. Pat. No. 9,539,218 B2 provides a tablet composition of rivaroxaban for treating thromboembolic disorders by administering the composition once daily for at least 5 consecutive days.

U.S. Pat. No. 9,415,053 B2 relates to fluidized bed granulation process for the preparation of a solid orally administrable pharmaceutical composition comprising rivaroxaban in hydrophilized form.

WO 2018001914 A1 discloses a pharmaceutical capsule composition comprising rivaroxaban with at least one pharmaceutically acceptable excipient, which mentions that using moisture or wet granulation techniques lead to stability problems. Therefore, they have developed the composition wherein powder mixture is processed without need of wet or moist granulation and is directly filled into capsules.

Therefore, there is still a need in the art to develop an alternate rivaroxaban capsule composition which overcomes the above described problems in prior art as well as offer advantages over them. Thus, the present inventors have developed simple, fast, cost-effective and industrially feasible process for the preparation of rivaroxaban compositions.

### SUMMARY OF INVENTION

According to one aspect of the present invention, there is provided a capsule composition comprising rivaroxaban and one or more pharmaceutically acceptable excipients.

According to another aspect of the present invention, there is provided a capsule composition comprising rivaroxaban and one or more pharmaceutically acceptable excipients selected from the group comprising diluent, binder, lubricant, glidant, surfactant, disintegrant and/or combinations or the like thereof.

According to another aspect of the present invention, there is provided a process for preparing a capsule composition comprising rivaroxaban and one or more pharmaceutically acceptable excipients, wherein said process comprises following steps:
a) sifting one or more pharmaceutically acceptable excipients to form powder blend;
b) preparing the drug suspension by adding drug, binder and one or more pharmaceutically acceptable excipients in a solvent;
c) granulating the powder blend with drug suspension in high shear granulator to obtain granules;
d) drying the granules followed by milling;
e) optionally lubricating the granules and optionally adding one or more pharmaceutically acceptable excipients; and
f) filling the granules in a capsule.

According to another aspect of the present invention, there is provided a capsule composition which is useful for the treatment of thromboembolic disorders.

The details of one or more embodiments of the inventions are set forth in the description below. Other features, objects and advantages of the inventions will be apparent from the description and claims.

### DETAILED DESCRIPTION OF INVENTION

The present invention relates to a capsule compositions comprising rivaroxaban or its pharmaceutically acceptable salt thereof and process for preparation thereof.

The term “Rivaroxaban” as used herein according to the present invention includes Rivaroxaban in the form of free base, a pharmaceutically acceptable salt thereof, amorphous rivaroxaban, crystalline rivaroxaban, any isomer, derivative, hydrate, solvate or prodrug or a combination thereof. In a preferred embodiment of the present invention, the rivaroxaban is employed in crystalline form.

The term “active ingredient” herein refers to a pharmaceutically active molecule as well as its pharmaceutically acceptable and therapeutically active salts, esters, amides, prodrugs, metabolites, enantiomers, polymorphs, analogues, etc. that induce a desired pharmacological or physiological effect. Terms like “active”, “active agent”, “active substance” may be used synonymously for “active ingredient”.

The term “excipient” or “pharmaceutically acceptable excipients” means pharmacologically inactive components that are useful in preparing dosage form and which are generally safe, non-toxic and are acceptable for veterinary as well as human pharmaceutical use. Reference to an excipient includes both one excipient and more than one excipient.

The term “stable or stability or stabilized” means the dosage form is stable under accelerated conditions (40° C./75% RH) and/or (25° C./60% RH) for at least 6 months.

The term “composition” or “solid oral composition” or “dosage form” or “pharmaceutical composition” or “formulation” as used herein synonymously include capsules, granules in a capsule, pellets, pellets in a capsule, powder, suspension or any other suitable dosage form meant for oral administration. Capsule is in the form of soft gelatin or hard gelatin or HPMC capsule.

According to one embodiment of the present invention, there is provided a capsule composition comprising rivaroxaban or pharmaceutically acceptable salts thereof and one or more pharmaceutically acceptable excipients, wherein the one or more pharmaceutically acceptable excipients are selected from the group comprising diluent, binder, lubricant, glidant, surfactant, disintegrant and/or combinations or the like thereof.

According to another embodiment of the present invention, there is provided a capsule composition comprising rivaroxaban and one or more pharmaceutically acceptable excipients, wherein the disintegrant and diluent are present in a ratio of 1:1 to 1:50, preferably in the ratio of 1:5 to 1:25, most preferably in the ratio of 1:5 to 1:15.

According to another embodiment of the present invention, there is provided a capsule composition comprising rivaroxaban and one or more pharmaceutically acceptable excipients, wherein the binder and diluent are present in a ratio of 1:1 to 1:50, preferably in the ratio of 1:1 to 1:40, most preferably in the ratio of 1:10 to 1:35.

According to another embodiment of the present invention, there is provided a process for preparing a capsule composition comprising rivaroxaban and one or more pharmaceutically acceptable excipients, wherein said process comprises following steps:
sifting one or more pharmaceutically acceptable excipients to form powder blend;
preparing the drug suspension by adding drug, binder and one or more pharmaceutically acceptable excipients in a solvent;
granulating the powder blend with drug suspension in high shear granulator to obtain granules;
drying the granules followed by milling;
optionally lubricating the granules and optionally adding one or more pharmaceutically acceptable excipients;
filling the granules in a capsule.

According to another embodiment of the present invention, the high shear granulator used to granulate the powder blend with drug suspension is preferably a rapid mixer granulator and the like.

According to another embodiment of the present invention, the capsule compositions can be prepared by any suitable method known in the art such as dry or wet granulation, extrusion spheronization, melt extrusion, melt granulation, spray coating, freeze drying, spray drying and solution evaporation. The coating may be applied as solution/dispersion of coating ingredients using any conventional coating techniques known in the art.

According to another embodiment of the present invention, there is provided a capsule composition comprising rivaroxaban or pharmaceutically acceptable salts thereof and one or more pharmaceutically acceptable excipients, wherein the capsule contains rivaroxaban coated pellets or granules.

According to another embodiment of the present invention, there is provided a capsule composition comprising granules of rivaroxaban wherein said granules comprises rivaroxaban and one or more pharmaceutically acceptable excipients selected from the group comprising diluent, binder, lubricant, glidant, surfactant, disintegrant and/or combinations or the like thereof.

According to another embodiment of the present invention, there is provided a capsule composition comprising rivaroxaban or pharmaceutically acceptable salts thereof and one or more pharmaceutically acceptable excipients, wherein the sugar spheres coated with rivaroxaban are filled into capsules.

According to another embodiment of the present invention, there is provided a capsule composition comprising rivaroxaban or pharmaceutically acceptable salts thereof and one or more pharmaceutically acceptable excipients, wherein the composition is free of glidant.

Also provided is a pharmaceutical composition, comprising a capsule of rivaroxaban and one or more pharmaceutically acceptable excipient(s) as provided herein, wherein at least 80% of rivaroxaban is released in 30 minutes upon administration to a subject or in vitro dissolution testing.

According to another embodiment of the present invention, the granules can be filled into capsules or sachets.

According to another embodiment of the present invention, there is provided granules comprising rivaroxaban and one or more pharmaceutically acceptable excipients selected from the group comprising diluent, binder, lubricant, glidant, surfactant, disintegrant and/or combinations or the like thereof.

Capsules can be soft gelatin capsules, hard gelatin capsules or HPMC capsules. Capsule shell may contain gelatin, HPMC, plasticizer or any other suitable pharmaceutically acceptable excipients. Gelatin capsules can be formed from animal gelatin or synthetic or plant derived equivalents thereof. In soft gelatin capsules, the active compound may be dissolved or suspended in suitable liquids, such as fatty oils, liquid paraffin, or liquid polyethylene glycols. The hard gelatin capsule, also known as the dry-filled capsule (DFC), consists of two sections, one slipping over the other. Other capsule materials may also be used and the selection thereof is within the purview of a person of ordinary skill in the art. However, HPMC based capsules are considered safe for normal human consumption instead of gelatin because of its vegetarian source. Further, capsule shell based on HPMC have better mechanical and tensile strength with improved shapability and disintegration time.

According to another embodiment of the present invention, capsule shell contains hydroxypropylmethyl cellulose, carrageenan, potassium chloride, titanium dioxide, and water.

According to another embodiment of the present invention, the bulk density of the rivaroxaban granules is in the range from about 0.1 g/ml to about 2 g/ml, preferably in the range of about 0.1 g/ml to about 1 g/ml.

According to another embodiment of the present invention, the tapped density of rivaroxaban granules is in range from about 0.1 g/ml to about 2 g/ml, preferably in the range of about 0.1 g/ml to about 1 g/ml.

According to another embodiment of the present invention, the mean particle size (D50) of rivaroxaban granules is in the range from about 5 μm to about 800 μm.

According to another embodiment of the present invention, the crystalline active rivaroxaban is employed in micronized form. The active rivaroxaban in this case preferably has an average particle size X₅₀ of less than 10 μm, in particular between 1 and 8 μm and X₉₀ of less than 20 μm, in particular of less than 15 μm.

According to another embodiment of the present invention, the composition is filled into hard gelatin capsules.

According to another embodiment of the present invention, disintegration time of hard gelatin capsules is 2 minutes to 10 minutes.

According to another embodiment of the present invention, the composition is filled into HPMC capsules.

According to another embodiment of the present invention, the capsule shell can be of the size ranging from size 000′ to size 5′.

Suitable diluents/fillers include but are not limited to starch, powdered celluloses, polysaccharides, dibasic calcium phosphate, calcium phosphate, calcium carbonate, calcium citrate, tricalcium citrate, magnesium carbonate, lactose monohydrate, lactose anhydrous, microcrystalline cellulose, mannitol, dextrose, sucrose, sorbitol, xylitol, inositol, dextrates, lactitol, maltodextrin, trehalose, and/or combinations or the like thereof. Further, the amount of diluent is preferably in the range of 10% to 90% by weight of the composition.

Suitable binders include but are not limited to starch, pregelatinized starch, acacia, gelatin, guar gum, partially hydrolyzed starch, alginates, xanthan gum, carbomers, alginic acid, glucose, gums, maltodextrin, polydextrose, polyethylene oxide, sodium alginate, corn starch, starch paste, sucrose and hydrophilic polymers such as, for example, hydroxypropylmethylcellulose (HPMC), carboxymethylcellulose (sodium and calcium salts), ethylcellulose, methylcellulose, hydroxyethylcellulose, ethylhydroxyethylcellulose, hydroxypropylcellulose (HPC), L-HPC (low-substituted HPC), microcrystalline cellulose, powdered cellulose, polyethylene glycol, polyvinylpyrrolidone, polyvinyl alcohol, polymers of acrylic acid and its salts, vinylpyrrolidone-vinyl acetate copolymers (for example Kollidon™. VA64, BASF) and/or combinations or the like thereof. Further, the amount of binder is present in a concentration of 0.1 to 25% based on the total weight of the pharmaceutical composition. Particularly preferably, HPMC is employed as a hydrophilic binding agent.

Suitable surfactants or wetting agents include but are not limited to anionic, cationic, non-ionic or amphoteric surfactants or those known to the person skilled in the art which includes sodium lauryl sulphate, docusate sodium acid, myristyl alcohol, cetrimide macrogol 15 hydroxystearate; sulphosuccinates such as sodium dioctyl sulphosuccinate; partial fatty acid esters of polyhydric alcohols such as glycerol monostearate, glyceryl monooleate; partial fatty acid esters of sorbitan such as sorbitan monolaurate, partial fatty acid esters of polyhydroxyethylenesorbitan such as polyethylene glycol monostearate or monooleate, polyhydroxyethylene fatty alcohol ethers, polyhydroxyethylene fatty acid esters, ethylene oxide-propylene oxide block copolymers (Pluronic™ or ethoxylated triglycerides. Preferably, sodium lauryl sulphate is employed as a wetting agent. Further, the amount of surfactant/wetting agents present in the dosages form of rivaroxaban or salt thereof ranges from about 0.1% to about 25% by total weight of the composition.

Suitable disintegrants include but are not limited to cross-linked carboxymethyl cellulose (croscarmellose sodium), cross-linked polyvinyl pyrrolidone (crospovidone), low-substituted hydroxypropyl cellulose, pregelatinized starch, sodium carboxymethyl cellulose, calcium carboxymethyl cellulose, carboxymethyl cellulose, docusate sodium, polyacryline potassium, sodium alginate, corn starch, pregelatinized starch, sodium starch glycolate, alginic acid, alginates, ion-exchange resins, magnesium aluminium silica, sodium glycine carbonate or combinations thereof. Further, the amount of disintegrant is preferably in the range of 0.1% to 25% by weight of the composition.

Suitable lubricants may comprise but not limited to magnesium stearate, calcium stearate, zinc stearate, talc, waxes, boric acid, hydrogenated vegetable oil, sodium chlorate, magnesium lauryl sulfate, sodium oleate, sodium acetate, sodium benzoate, polyethylene glycol, stearic acid, fatty acid, fumaric acid, glyseryl palmito sulphate, sodium stearyl fumarate and/or combinations or the like thereof. Further, the amount of lubricant is preferably in the range of 0.1% to 20% by weight of the composition.

Suitable glidants may comprise but not limited to talc, colloidal silicon dioxide, aluminum silicate, colloidal silica, starch and/or combinations or the like thereof. Further, the amount of glidant is preferably in the range of 1% to 50% by weight of the composition.

Suitable solvents include but are not limited to, water, organic solvents, inorganic solvents and/or combinations thereof.

According to another embodiment of the present invention, there is provided a capsule composition which is useful for the treatment of thromboembolic disorders.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1: Capsule Composition of Rivaroxaban

| | | | | |
|---|---|---|---|---|
| Ingredient | 2.5 mg | 10 mg | 15 mg | 20 mg |

Drug Suspension

| | | | | |
|---|---|---|---|---|
| Rivaroxaban | 2.50 | 10.00 | 15.00 | 20.00 |
| Sodium Lauryl Sulfate | 0.50 | 0.50 | 0.75 | 1.00 |
| Polyvinylpyrrolidone | 2.50 | 2.50 | 3.75 | 5.00 |
| Purified Water | q.s. | q.s. | q.s. | q.s. |

Intragranular material

| | | | | |
|---|---|---|---|---|
| Lactose Monohydrate | 38.42 | 40.00 | 60.00 | 80.00 |
| Microcrystalline | 32.48 | 23.40 | 35.10 | 46.80 |
| Cellulose | | | | |
| Croscarmellose Sodium | 3.00 | 3.00 | 4.50 | 6.00 |

Lubrication material

| | | | | |
|---|---|---|---|---|
| Magnesium Stearate | 0.60 | 0.60 | 0.90 | 1.20 |
| Average Fill weight | 80.00 | 80.00 | 120.00 | 160.00 |
| EHG (Empty Hard | 5 | 5 | 4 | 3 |
| Gelatin) Capsule Size | | | | |

Brief Manufacturing Process:

1. Sifting:
Sift together microcrystalline cellulose, lactose monohydrate and croscarmellose sodium through suitable sieve.
Sift rivaroxaban through suitable sieve.
Sift magnesium stearate through suitable sieve.

2. Drug Suspension:
Dissolve polyvinylpyrrolidone and sodium lauryl sulphate in water.
Suspend rivaroxaban in the above solution to form drug suspension.

3. Granulation:
Load the sifted microcrystalline cellulose, lactose and croscarmellose sodium in rapid mixer granulator.
Perform the granulation using drug suspension in rapid mixer granulator.

4. Drying and Milling:
Dry the granules in dryer and mill the dried granules.

5. Lubrication:
Lubricate the milled granules with magnesium stearate.

6. Capsule Filling:
Fill the lubricated blend in suitable capsule shell.

### Example 2: Capsule Composition of Rivaroxaban

| | | | | |
|---|---|---|---|---|
| Ingredient | 2.5 mg | 10 mg | 15 mg | 20 mg |

Drug Suspension

| | | | | |
|---|---|---|---|---|
| Rivaroxaban | 2.50 | 10.00 | 15.00 | 20.00 |
| Sodium Lauryl Sulfate | 0.34 | 0.50 | 0.75 | 1.00 |
| Hydroxypropyl cellulose | 4.505 | 6.755 | 10.13 | 13.51 |
| Purified Water | q.s. | q.s. | q.s. | q.s. |

Intragranular material

| | | | | |
|---|---|---|---|---|
| Lactose Monohydrate | 18.12 | 10.88 | 16.32 | 21.76 |
| Microcrystalline Cellulose | 25.96 | 49.51 | 74.27 | 99.02 |
| Croscarmellose Sodium | 3.62 | 5.00 | 7.50 | 10.00 |
| Hydroxypropyl cellulose | 4.505 | 6.755 | 10.13 | 13.51 |

Lubrication material

| | | | | |
|---|---|---|---|---|
| Magnesium Stearate | 0.45 | 0.60 | 0.90 | 1.20 |
| Average Fill weight | 60.00 | 90.00 | 135.00 | 180.00 |
| EHG Capsule Size | 5 | 4 | 4 | 3 |

Brief Manufacturing Process:

1. Sifting:
Sift together microcrystalline cellulose, lactose monohydrate and croscarmellose sodium hydroxypropyl cellulose through suitable sieve.
Sift rivaroxaban through suitable sieve.
Sift magnesium stearate through suitable sieve.

2. Drug Suspension:
Dissolve hydroxypropyl cellulose and sodium lauryl sulphate in water.
Suspend rivaroxaban in the above solution to form drug suspension.

3. Granulation:
Load the sifted microcrystalline cellulose, lactose, hydroxypropyl cellulose and croscarmellose sodium in rapid mixer granulator.
Perform the granulation using drug suspension in rapid mixer granulator.

4. Drying and Milling:
Dry the granules in dryer and mill the dried granules.

5. Lubrication:
Lubricate the milled granules with magnesium stearate.

6. Capsule Filling:
Fill the lubricated blend in suitable capsule.

### Example 3: Capsule Composition of Rivaroxaban

| | | | | |
|---|---|---|---|---|
| Ingredient | 2.5 mg | 10 mg | 15 mg | 20 mg |

Drug Suspension

| | | | | |
|---|---|---|---|---|
| Rivaroxaban | 2.50 | 10.00 | 15.00 | 20.00 |
| Sodium Lauryl Sulfate | 0.34 | 0.50 | 0.75 | 1.00 |
| Hydroxypropyl methyl | 4.505 | 6.755 | 10.13 | 13.51 |
| cellulose | | | | |
| Purified Water | q.s. | q.s. | q.s. | q.s. |

Intragranular material

| | | | | |
|---|---|---|---|---|
| Lactose Monohydrate | 18.12 | 10.88 | 16.32 | 21.76 |
| Microcrystalline Cellulose | 25.96 | 49.51 | 74.27 | 99.02 |
| Croscarmellose Sodium | 3.62 | 5.00 | 7.50 | 10.00 |
| Hydroxypropyl methyl | 4.505 | 6.755 | 10.13 | 13.51 |
| cellulose | | | | |

Lubrication material

| | | | | |
|---|---|---|---|---|
| Magnesium Stearate | 0.45 | 0.60 | 0.90 | 1.20 |
| Average Fill weight | 60.00 | 90.00 | 135.00 | 180.00 |
| EHG Capsule Size | 5 | 4 | 4 | 3 |

Brief Manufacturing Process:

1. Sifting:
Sift together microcrystalline cellulose, lactose monohydrate and croscarmellose sodium and hydroxypropyl methyl cellulose through suitable sieve.
Sift rivaroxaban through suitable sieve.
Sift magnesium stearate through suitable sieve.

2. Drug Suspension:
Dissolve hydroxypropyl methyl cellulose and sodium lauryl sulphate in water.
Suspend rivaroxaban in the above solution to prepare drug suspension.

3. Granulation:
Load the sifted microcrystalline cellulose, lactose, hydroxypropyl methyl cellulose and croscarmellose sodium in fluidized bed processor.
Perform the granulation using drug suspension in fluidized bed processor.

4. Drying and Milling:
Dry the granules and then mill the dried granules.

5. Lubrication:
Lubricate the milled granules with sifted magnesium stearate.

6. Capsule Filling:
Fill the lubricated blend in suitable capsule shell.

### Example 4: Capsule Composition of Rivaroxaban

| | | | | |
|---|---|---|---|---|
| Ingredient | 2.5 mg | 10 mg | 15 mg | 20 mg |

Drug Suspension

| | | | | |
|---|---|---|---|---|
| Rivaroxaban | 2.50 | 10.00 | 15.00 | 20.00 |
| Sodium Lauryl Sulfate | 0.34 | 0.50 | 0.75 | 1.00 |
| Hydroxypropyl methyl cellulose | 4.505 | 6.755 | 10.13 | 13.51 |
| Purified Water | q.s. | q.s. | q.s. | q.s. |

Intragranular material

| | | | | |
|---|---|---|---|---|
| Lactose Monohydrate | 18.12 | 10.88 | 16.32 | 21.76 |
| Microcrystalline Cellulose | 25.96 | 49.51 | 74.27 | 99.02 |
| Maize Starch | 3.62 | 5.00 | 7.50 | 10.00 |
| Hydroxypropyl methyl cellulose | 4.505 | 6.755 | 10.13 | 13.51 |

Lubrication material

| | | | | |
|---|---|---|---|---|
| Colloidal Silicon dioxide | 0.45 | 0.60 | 0.90 | 1.20 |
| Average Fill weight | 60.00 | 90.00 | 135.00 | 180.00 |
| EHG Capsule Size | 5 | 4 | 4 | 3 |

Brief Manufacturing process:

1. Sifting:
Sift together microcrystalline cellulose, lactose monohydrate and maize starch and hydroxypropyl methyl cellulose through suitable sieve.
Sift rivaroxaban through suitable sieve.
Sift colloidal silicon dioxide through suitable sieve.

2. Drug Suspension:
Dissolve hydroxypropyl methyl cellulose and sodium lauryl sulphate in water.
Suspend rivaroxaban in the above solution to form drug suspension

3. Granulation:
Load the sifted microcrystalline cellulose, lactose, hydroxypropyl methyl cellulose and maize starch in fluidized bed processor.
Perform the granulation using drug suspension in fluidized bed processor.

4. Drying and Milling:
Dry the granules and mill the dried granules.

5. Lubrication:
Lubricate the milled granules with colloidal silicon dioxide.

6. Capsule Filling:
Fill the lubricated blend in suitable capsule shell.

### Example 5: Capsule Composition of Rivaroxaban

| | | | | |
|---|---|---|---|---|
| Ingredient | 2.5 mg | 10 mg | 15 mg | 20 mg |

Drug Suspension

| | | | | |
|---|---|---|---|---|
| Rivaroxaban | 2.50 | 10.00 | 15.00 | 20.00 |
| Sodium Lauryl Sulfate | 0.88 | 1.50 | 1.50 | 1.50 |
| Hydroxypropyl methyl cellulose | 0.71 | 1.20 | 1.20 | 1.20 |
| Purified Water | q.s. | q.s. | q.s. | q.s. |

Intragranular material

| | | | | |
|---|---|---|---|---|
| Lactose Monohydrate | 35.00 | 51.30 | 51.30 | 51.30 |
| Microcrystalline Cellulose | 49.75 | 75.50 | 70.50 | 65.50 |
| Croscarmellose Sodium | 1.76 | 3.00 | 3.00 | 3.00 |
| Hydroxypropyl methyl cellulose | 1.76 | 3.00 | 3.00 | 3.00 |

Extragranular material

| | | | | |
|---|---|---|---|---|
| Microcrystalline Cellulose | 5.00 | 20.00 | 20.00 | 20.00 |
| Croscarmellose Sodium | 1.76 | 3.00 | 3.00 | 3.00 |

Lubrication material

| | | | | |
|---|---|---|---|---|
| Magnesium Stearate | 0.88 | 1.50 | 1.50 | 1.50 |
| Average Fill weight | 100.00 | 170.00 | 170.00 | 170.00 |
| EHG Capsule Size | 4 | 3 | 3 | 3 |

Brief Manufacturing Process:

1. Sifting:
Sift together microcrystalline cellulose, lactose monohydrate, hydroxypropyl methyl cellulose and croscarmellose sodium through suitable sieve.
Sift rivaroxaban through suitable sieve.
Sift magnesium stearate through suitable sieve.

2. Drug Suspension:
Dissolve hydroxypropyl methyl cellulose and sodium lauryl sulfate in purified water.
Suspend rivaroxaban in the above solution to form drug suspension.

3. Granulation:
Load the intragranular materials in rapid mixer granulator.
Perform the granulation using drug suspension in rapid mixer granulator.

4. Drying and Milling:
Dry the granules in dryer and mill the dried granules.

5. Pre-Lubrication:
Pre-lubricate the milled granules with microcrystalline cellulose and croscarmellose sodium.

6. Lubrication:
Lubricate the pre-lubricated granules in suitable blender with magnesium stearate.

7. Capsule Filling:
Fill the lubricated blend in suitable capsule shell.

### Example 6: Capsule Composition of Rivaroxaban

| | | | | |
|---|---|---|---|---|
| Ingredient | 2.5 mg | 10 mg | 15 mg | 20 mg |

Drug Suspension

| | | | | |
|---|---|---|---|---|
| Rivaroxaban | 2.50 | 10.00 | 15.00 | 20.00 |
| Hydroxypropyl methyl cellulose | 0.71 | 1.20 | 1.20 | 1.20 |
| Isopropyl alcohol | q.s. | q.s. | q.s. | q.s. |

Intragranular material

| | | | | |
|---|---|---|---|---|
| Lactose Monohydrate | 35.00 | 51.30 | 51.30 | 51.30 |
| Microcrystalline Cellulose | 49.75 | 75.50 | 70.50 | 65.50 |
| Croscarmellose Sodium | 1.76 | 3.00 | 3.00 | 3.00 |
| Sodium Lauryl Sulfate | 0.88 | 1.50 | 1.50 | 1.50 |
| Hydroxypropyl methyl cellulose | 1.76 | 3.00 | 3.00 | 3.00 |

Extragranular material

| | | | | |
|---|---|---|---|---|
| Microcrystalline Cellulose | 5.00 | 20.00 | 20.00 | 20.00 |
| Croscarmellose Sodium | 1.76 | 3.00 | 3.00 | 3.00 |

Lubrication material

| | | | | |
|---|---|---|---|---|
| Magnesium Stearate | 0.88 | 1.50 | 1.50 | 1.50 |
| Average Fill weight | 100.00 | 170.00 | 170.00 | 170.00 |
| EHG Capsule Size | 4 | 3 | 3 | 3 |

Brief Manufacturing Process:

1. Sifting:
Sift together microcrystalline cellulose, lactose monohydrate, hydroxypropyl methyl cellulose, sodium lauryl sulfate and croscarmellose sodium through suitable sieve.
Sift rivaroxaban through suitable sieve.
Sift magnesium stearate through suitable sieve.

2. Drug Suspension:
Dissolve hydroxypropyl methyl cellulose in isopropyl alcohol.

Suspend rivaroxaban in the above solution.

3. Granulation:
Granulate the intragranular materials in rapid mixer granulator using drug suspension.

4. Drying and Milling:
Dry the granules in fluid bed dryer and mill the dried granules.

5. Pre-Lubrication:
Pre-lubricate the milled granules in suitable blender with co-sifted microcrystalline cellulose and croscarmellose sodium.

6. Lubrication:
Lubricate the pre-lubricated granules in suitable blender with sifted magnesium stearate.

7. Capsule Filling:
Fill the lubricated blend in suitable capsule.

### Example 7: Capsule Composition of Rivaroxaban

| | | | | |
|---|---|---|---|---|
| Ingredient | 2.5 mg | 10 mg | 15 mg | 20 mg |

Drug Suspension

| | | | | |
|---|---|---|---|---|
| Rivaroxaban | 2.50 | 10.00 | 15.00 | 20.00 |
| Sodium Lauryl Sulfate | 0.75 | 0.75 | 1.125 | 1.50 |
| Hydroxypropyl methyl cellulose | 0.60 | 0.60 | 0.90 | 1.20 |
| Purified Water | q.s. | q.s. | q.s. | q.s. |

Intra-granular material

| | | | | |
|---|---|---|---|---|
| Lactose Monohydrate | 31.00 | 25.65 | 38.475 | 51.30 |
| Microcrystalline Cellulose | 25.14 | 23.00 | 34.500 | 46.00 |
| Croscarmellose Sodium | 4.00 | 4.00 | 6.00 | 8.00 |
| Hydroxypropyl methyl cellulose | 1.50 | 1.50 | 2.25 | 3.00 |

Pre-lubrication Material

| | | | | |
|---|---|---|---|---|
| Microcrystalline Cellulose | 10.00 | 10.00 | 15.00 | 20.00 |
| Croscarmellose Sodium | 4.00 | 4.00 | 6.00 | 8.00 |

Lubrication material

| | | | | |
|---|---|---|---|---|
| Magnesium Stearate | 0.50 | 0.50 | 0.75 | 1.00 |
| Average Fill weight | 80.00 | 80.000 | 120.00 | 160.00 |
| HPMC Capsule Size | 4 | 4 | 3 | 3 |

Brief Manufacturing Process:

1. Sifting:
Sift together intra-granular materials through suitable sieve.
Sift together pre-lubrication materials through suitable sieve.
Sift magnesium stearate through suitable sieve.

2. Drug Suspension (Binder):
Dissolve hydroxypropyl methyl cellulose and sodium lauryl sulphate in purified water.
Suspend rivaroxaban in the above solution to form drug suspension.

3. Granulation:
Load the sifted intra-granular materials in rapid mixer granulator.
Perform granulation using drug suspension.

4. Drying and Milling:
Dry the granules in fluid bed dryer, mill the dried granules and then sift the milled granules.

5. Blending, Pre-lubrication & Lubrication:
Lubricate the granules with pre-lubrication and lubrication materials.

6. Capsule Filling:
Fill the lubricated blend in suitable capsule.

Dissolution studies Drug dissolution studies of the various strengths of the pharmaceutical composition of Example 7 were carried out using USP dissolution apparatus II (Paddle) at 75 rpm and at temperature of 37°±0.5° C. in 900 mL of dissolution medium. The results are reported for dissolution medium pH 4.5 acetate buffer in Table 1 (for 2.5 mg), for dissolution medium pH 4.5 Acetate Buffer+0.2% SDS in Table 2 (for 10 mg) and for dissolution medium pH 4.5 Acetate Buffer+0.4% SDS in Table 3 (for 15 mg and 20 mg) respectively.

TABLE 1
Percentage drug (Rivaroxaban) release for 2.5 mg
pH 4.5 Acetate Buffer, Volume: 900 mL, RPM* 75, Apparatus
USP-II (Paddle)

| Time Points | Xarelto ™ (Tablet) | Example 7 |
|---|---|---|
| (mins) | % Release | % Release |
| 10 | 84 | 88 |
| 15 | 88 | 98 |
| 20 | 90 | 99 |
| 30 | 93 | 100 |
| 45 | 95 | 101 |
| 60 | 96 | 101 |

*RPM is Rotation Per Minute

TABLE 2
Percentage drug (Rivaroxaban) release for 10 mg
pH 4.5 Acetate Buffer + 0.2% SDS, Volume: 900 mL, RPM 75,
Apparatus USP-II (Paddle)

| Time Points | Xarelto ™ (Tablet) | Example 7 |
|---|---|---|
| (mins) | % Release | % Release |
| 10 | 87 | 97 |
| 15 | 90 | 99 |
| 20 | 92 | 100 |
| 30 | 93 | 101 |
| 45 | 93 | 102 |
| 60 | 94 | 103 |

TABLE 3
Percentage drug (Rivaroxaban) release for 15 mg & 20 mg
pH 4.5 Acetate Buffer + 0.4% SDS, Volume: 900 mL, RPM 75,
Apparatus USP-II (Paddle)

| Time | Xarelto ™ | Example 7 | Example 7 |
|---|---|---|---|
| Points | (Tablet) | For 15 mg | For 20 mg |
| (mins) | % Release | % Release | % Release |
| 10 | 83 | 83 | 85 |
| 15 | 88 | 89 | 95 |
| 20 | 91 | 90 | 96 |
| 30 | 96 | 91 | 96 |
| 45 | 97 | 91 | 97 |
| 60 | 97 | 92 | 98 |

Stability Studies

The pharmaceutical composition of Example 7 was subjected to stability studies at 40° C./75% RH and 25° C./60% RH for 3 months; and the composition was found to be stable as per the data shown below in Table 4.

TABLE 4
Stability Study data

| | 40° C./75% RH* | | | 25° C./60% RH* |
|---|---|---|---|---|
| | Percentage drug Assay | | | |
| | Initial | 1.5 Months | 3 Months | 3 Months |
| For 2.5 mg | 101.1% | 101.9% | 101.9% | 101.7% |
| For 10 mg | 100.4% | 101.3% | 101.9% | 100.8% |
| For 15 mg | 99.9% | 100.1% | 100% | 99.9% |
| For 20 mg | 99.1% | 99.9% | 101.1% | 101.5% |

*Relative Humidity

Impurity Data

The various strengths of the pharmaceutical composition of Example 7 were subjected to impurity testing at 40° C./75% RH and 25° C./60% RH for 3 months; and the impurity data is represented below in Table 5 and Table 6 respectively. Impurity B is (S)-4-[4-[5-(Aminomethyl)-2-oxo-oxazolidin-3-yl]phenyl]morpholin-3-one; Impurity D is (S)-2-[[2-oxo-3-[4-(3-oxo-morpholino)phenyl]oxazolidine-5-yl]-methyl]isoindoline-1,3-dione; Impurity A is 5-Chlorothiophene-2-Carboxylic acid; impurity C is (R)-4-[4-(3-Phthalimido-2-hydroxypropylamino)phenyl]morpholin-3-one.

TABLE 5
Impurity data

| | 2.5 mg | | | 10 mg | | |
|---|---|---|---|---|---|---|
| | | 40° C./ | 25° C./ | | 40° C./ | 25° C./ |
| | | 75% | 60% | | 75% | 60% |
| | | RH* | RH* | | RH* | RH* |
| Impurity | Initial | 3 Months | 3 Months | Initial | 3 Months | 3 Months |
| Impurity B | ND | ND | ND | ND | ND | ND |
| Impurity D | 0.05 | 0.05 | 0.05 | 0.05 | 0.04 | 0.04 |
| Impurity A | 0 | ND | 0 | ND | ND | ND |
| Impurity C | ND | ND | ND | ND | ND | ND |
| Any highest | 0.00 | 0.05 | 0.05 | ND | 0.05 | 0.05 |
| unspecified | | | | | | |
| degradation | | | | | | |
| product | | | | | | |
| Total | 0.05 | 0.10 | 0.10 | 0.05 | 0.09 | 0.09 |
| impurities | | | | | | |

*Relative Humidity

TABLE 6
Impurity data

| | 15 mg | | | 20 mg | | |
|---|---|---|---|---|---|---|
| | | 40° C./ | 25° C./ | | 40° C./ | 25° C./ |
| | | 75% | 60% | | 75% | 60% |
| | | RH* | RH* | | RH* | RH* |
| Impurity | Initial | 3 Months | 3 Months | Initial | 3 Months | 3 Months |
| Impurity B | ND | ND | ND | ND | ND | ND |
| Impurity D | 0.04 | 0.04 | 0.04 | 0.04 | 0.04 | 0.04 |
| Impurity A | ND | ND | ND | ND | ND | ND |
| Impurity C | ND | ND | ND | ND | ND | ND |
| Any highest | 0.00 | 0.05 | 0.04 | 0.00 | 0.05 | 0.05 |
| unspecified | | | | | | |
| degradation | | | | | | |
| product | | | | | | |
| Total | 0.04 | 0.09 | 0.08 | 0.04 | 0.09 | 0.09 |
| impurities | | | | | | |

*Relative Humidity

The above impurity data reflects that the total impurities in the pharmaceutical composition of the present invention is within limits.

Many modifications and variations of the embodiments described herein may be made without departing from the scope, as is apparent to those skilled in the art. The specific embodiments described herein are offered by way of example only.

We claim:

1. A capsule composition comprising rivaroxaban or its pharmaceutically acceptable salts, ester, solvates, polymorphs thereof and one or more pharmaceutically acceptable excipients.

2. The capsule composition as claimed in claim 1, wherein said pharmaceutically acceptable excipients are selected from the group comprising diluent, binder, lubricant, glidant, surfactant, disintegrant and/or combinations thereof.

3. The capsule composition as claimed in claim 1, wherein said disintegrant and diluent are present in a ratio of 1:1 to 1:50.

4. The capsule composition as claimed in claim 1, wherein said binder and diluent are present in a ratio of 1:1 to 1:50.

5. The capsule composition as claimed in claim 1, wherein said composition comprises granules of rivaroxaban having mean particle size in the range from about 5 μm to about 800 μm.

6. The capsule composition as claimed in claim 1, wherein said composition comprises granules of rivaroxaban having bulk density in the range from about 0.1 g/ml to about 2 g/ml.

7. The capsule composition as claimed in claim 1, wherein said composition comprises granules of rivaroxaban having tapped density in the range from about 0.1 g/ml to about 2 g/ml.

8. The capsule composition as claimed in claim 1, wherein said pharmaceutical composition releases at least 80% of rivaroxaban in about 30 minutes.

9. The capsule composition as claimed in claim 1, wherein said pharmaceutical composition is free of glidant.

10. A process for preparing capsule composition, wherein said process comprises the step of:
a) sifting one or more pharmaceutically acceptable excipients to form powder blend;
b) preparing the drug suspension by adding drug, binder and one or more pharmaceutically acceptable excipients in a solvent;
c) granulating the powder blend with drug suspension in high shear granulator to obtain granules;
d) drying the granules followed by milling;
e) optionally lubricating the granules and optionally adding one or more pharmaceutically acceptable excipients; and
f) filling the granules in a capsule.
